(19) United States
(12) Patent Application Publication
(10) Pub. No.: US 2023/0237926 A1
(43) Pub. Date: Jul. 27, 2023

(54) Cognitive Training Using Voice Command

(71) Applicant: Lumos Labs, Inc., San Francisco, CA (US)

(72) Inventors: Aaron Kaluszka, Hayward, CA (US); Robert John Schafer, El Macero, CA (US)

(21) Appl. No.: 18/100,770

(22) Filed: Jan. 24, 2023

Related U.S. Application Data
(60) Provisional application No. 63/302,393, filed on Jan. 24, 2022.

(51) Int. Cl. G09B 7/02; G06F 3/04842; G10L 15/18; G10L 15/22; G09B 19/02; G16H 40/20

(52) U.S. Cl. CPC G09B 7/02; G06F 3/04842; G10L 15/1822; G10L 15/22; G10L 15/1807; G09B 19/025; G16H 40/20; G10L 2015/223; G16H 10/20

(57) ABSTRACT

Systems and methods for cognitive training using voice command are described. One aspect includes a device to repeatedly present visual stimuli to a user that require performance of a task. A microphone may be positioned to provide audio input from the user to the device, with the audio input from the user providing input required to measure task performance. A processing system may perform real-time analysis of measured task performance.

### RELATED APPLICATION

The present disclosure is part of a non-provisional patent application claiming the priority benefit of U.S. Patent Application No. 63/302,393, filed on Jan. 24, 2022, which is incorporated by reference in its entirety.

### FIELD OF THE INVENTION

This invention relates generally to the field of cognitive training, including providing voice-based user tasks with measurable results. In some embodiments, gameplay or task repetition provides measurable improvement in cognitive skills.

### BACKGROUND

In many existing games that provide cognitive training, configurations of visual and/or auditory stimuli are presented to the user, along with rules for how to correctly complete a task. For example, a grid of squares can be displayed on a screen, and a subset of those squares briefly shown in a different color. The rules of the task indicate that a user must recall the locations of the colored squares in order to correctly complete the task. Successful completion of the task requires one or more cognitive skills such as recalling the locations of colored squares. In one example, repeated gameplay or task performance can improve short-term visuospatial memory.

In many existing games a user indicates his or her responses through a keyboard, mouse, or touchscreen (keyboard button presses, mouse movements, mouse clicks, screen taps, and/or screen swipes). By interacting with the games in this manner over the course of many repetitions, the user improves at the cognitive skills that were required to translate the stimuli to a correct response given the stated task rules.

### BRIEF DESCRIPTION OF THE DRAWINGS

The specific features, aspects and advantages of the present invention will become better understood with regard to the following description and accompanying drawings where:

FIG. 1 illustrates a system allowing cognitive training using speech based responses with real time feedback; and

FIG. 2 illustrates a method of cognitive training using speech based responses with real time feedback.

### DETAILED DESCRIPTION

In the following disclosure, reference is made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In some embodiments, a cognitive training system includes a device to repeatedly present visual, audio, or other stimuli to a user that require performance of a task. A microphone can be positioned to provide audio input from the user to the device, with the audio input from the user providing input required to measure task performance. Real time analysis of measured task performance can be provided to the user.

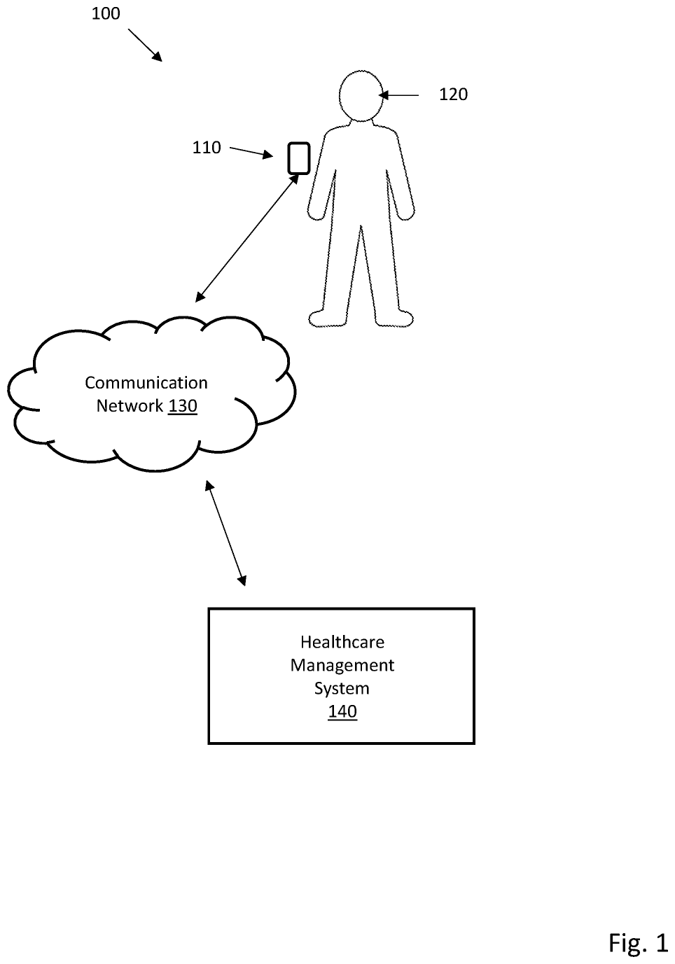

FIG. 1 illustrates a system 100 that can include a cognitive training device 110 with at least one of visual, audio, tactile, or other user 120 detectible output, and audio and/or tactile user input. The cognitive training device 110 can include a microphone to receive voice or speech input and provide a platform for operation of a cognitive game that accepts voice response. The device 110 can process data locally, via remote server, or through a combination of local and remote processing. In some embodiments the device 110 can be connected to a remote communication network 130 through wired or wireless connection. In some embodiments, user input data and other information can be transmitted to a healthcare management system 140 for further analysis or to allow for preservation of user data history.

In one embodiment, when the user speaks to the device 110 to indicate his or her responses, a computer microphone receives the sound of the speech. Software parses the speech into words. The cognitive game determines whether the spoken words correspond to legal task responses, or to other cognitive game commands (e.g., “Quit”, “Pause”). If a word corresponds to a task response or other predefined cognitive game command, the cognitive game acknowledges and processes the user input. If the interpreted word is ambiguous, then the cognitive game takes appropriate steps (e.g., choosing the interpretation that makes most sense in context of the cognitive game state, ignoring the word, or asking for clarification).

In such voice-based cognitive training games, stimuli can include visual and/or auditory cues, a stated rule indicating how to correctly complete the task, completion of the task requires one or more cognitive skills, and repeated use of the cognitive game improves the required cognitive skills.

In one instantiation, the set of legal user responses is a small and finite set (e.g. {yes, no}, {true, false}, {now}, or {up, down, left, right}). Instead of indicating these responses through keyboard, mouse, or screen interactions, the user speaks the response (e.g., “yes”). For example, a task requires responding to the orientation (up, down, left, or right) of a target stimulus while ignoring any distractors. In voice-based training, a user would achieve this by speaking the correct direction word.

In another instantiation, the goal of the task is to come up with a word that fits all of the requirements of the stated rules of the task. Instead of typing the word on a keyboard or touchscreen, the user speaks the response. For example, a task requires generating many words that start with a stem shown on the screen (for example “str”). A correct response could be given by saying the word “strong”.

In another instantiation, a goal of the task is to respond with a number that fits all of the requirements of the stated rules of the task. Instead of typing the number on a keypad or touchscreen, the user speaks the name of the number. For example, a task requires quickly and correctly solving arithmetic problems shown on the screen. A correct answer could be given by saying the appropriate number, for example “fourteen.”

In another instantiation, a goal of the task is to respond with the location of one or more stimuli on the screen. In a voice version of such a task, locations can be labeled for reference (for example with numbers or letters). Instead of clicking or tapping on screen locations, the user speaks the label associated with the location. For example, a user must interact with a set of switches that change the paths of trains going down a forking railroad track. These switches could be numbered, and the user would change the track configuration at a switch by saying the number of the desired switch, for example “five”.

In another instantiation, the goal of a task is to respond with the identity of a particular visual or auditory stimulus that is shown on the screen. Instead of selecting a stimulus with a mouse, keyboard, or touchscreen, the user names the stimulus itself, or an associated label. For example, a user is tasked with selecting items on a beach that have not been selected previously during the cognitive game session. An item could be selected by saying the object's name, for example “beachball.” In another example, each eligible item might be labeled with a number or letter. A user could select an item by saying the associated label, for example “C”.

In another instantiation, the goal of the task is to direct actions to take given a configuration shown on the screen. For example, a user must navigate a taxi to pick up and drop off passengers at their destinations. A user could say “pick up the cat” or drop off the purple dog”.

In another instantiation, the goal of the task is to spell a word. The user could say each letter of the desired word, in order. For example, a user must unscramble a set of letters shown on the screen to identify a word that matches the definition shown. For a clue “animal with four legs” and the scrambled letters “ATC,” the user could speak “C-A-T” to correctly spell the word “CAT.”

Advantageously, voice-based brain training may improve the engagement and/or effectiveness of training. Voice responses can make cognitive training tasks accessible to those who are physically unable to perform manual keyboard, mouse, or touchscreen interactions. For example, a person with severe arthritis may find manual interactions prohibitive, whereas voice interactions would make brain training possible.

In addition, use of voice can make brain training more convenient in contexts in which it is difficult to have manual interaction with a computer, thus allowing for more frequent training. For example, a user could do their brain training even when their hands are full or otherwise occupied.

As another advantage, because many human behaviors are deeply rooted in language, speech is a more natural interaction than keyboard button presses. Users may find that speech input makes cognitive games feel more immersive and fun.

In some embodiments, because speech is a more natural interaction than the use of computer peripherals or touchscreens, a user may improve cognitive skills in a context that is more likely to generalize to other things in their lives, i.e., the learning can transfer to a greater extent.

In verbal fluency cognitive games involving the generation of words, the action of producing speech may itself improve fluency because hearing the spoken responses may lead the user to think of more words with similar sounds.

In response inhibition cognitive games involving the identification of a conflicting stimulus where immediate response is paramount, a response in the form of speech may be necessary whereas selecting from a multiple choice or free input answer via physical methods would take too long, thus dampening the response inhibition effect.

In still other embodiments, a “phonological loop,” is formed in which language is held in a short-term memory store. Subvocal rehearsal of the stored language can work more or less efficiently when the final cognitive game response is given through speech instead of typing.

In some embodiments, a cognitive game also includes voice commands for the control of cognitive game operation itself, enabling the user to navigate the cognitive training program—i.e., play through a sequence of cognitive games—using voice commands. In such embodiments, games and training sessions are accessed through a central interface, which can appear on dedicated voice devices or from PCs or mobile devices. Performance data can be shown after each cognitive game play and can be transmitted through generated speech. Users can link accounts in order to synchronize progress, data, and personalized recommendations.

In addition to receiving and recognizing speech as commands/inputs, generate speech generation can be used to provide user input. This allows extension of types of stimuli used in the training tasks. For example, a novel training task can be generated that involves two-way conversation. For example, the cognitive game speaks to the user and the cognitive game's speech requires the user to exercise a cognitive skill or skills to generate a correct response given a stated rule, and the response is given via the user's voice.

Since speech is a rich signal, other features can be extracted from the speech besides the meaning of the user's words. The rules of a cognitive game could require the user to produce other qualities of speech (e.g., prosody, pace) rather than words with the correct meaning.

As will be understood, a user can be a human or non-human of any age and physical or mental capacity.

A cognition training device can be any of a wide variety of computing devices, such as a smart watch, a wearable device, smartphone, a desktop computer, a notebook computer, a tablet computer, a server computer, a handheld computer, imagers, digital cameras, and the like. The device 110 can include I/O device(s) include various devices that allow data and/or other information to be input to or retrieved. Example I/O device(s) include cursor control devices, keyboards, keypads, microphones, monitors or other display devices, speakers, printers, or network interface cards, modems, lenses, CCDs or other image capture devices, and the like. Device 110 can also include various interfaces that allow interaction with other systems, devices, or computing environments. For example, device 110 can include any number of different network interfaces, such as interfaces to local area networks (LANs), wide area networks (WANs), wireless networks, satellite networks, or other suitable internet or direct connection systems.

In some embodiments, device 110 includes one or more processors and processing support modules such as data busses, data oracles, smart contracts, memory device(s), mass storage device(s), decentralized ledger(s), and I/O device(s) to communicate with one other processing support modules, as well as other coupled devices. Busses can include one or more of several types of bus structures, such as a system bus, graphics bus, PCI bus, IEEE 1394 bus, or a USB bus. Using the processors and processing support modules, device 110 can execute programs or applications to provide for data capture, receipt, analysis, and transmission.

The device 110 can be connected to the communication network 130 but can also work independent of connection. Communication network 130 can include any type of network topology using any communication protocol. Additionally, data communication network 130 may include a combination of two or more communication networks. In some embodiments, data communication network 130 includes a cellular communication network, the Internet, a local area network, a wide area network, satellite networks, other suitable internet or direct connection systems, or any other communication network.

The healthcare management system 140 can be one or more systems that individually or collectively provide medical data collection and analysis services, along with support for remote clinical trials and clinical care. Hardware supporting operation of the healthcare management system 140 can be similar to that discussed with respect device 110, but can further include use of interconnected computing devices, including one or more of server, desktop, or laptop computers. Interconnect can be through different network interfaces, such as interfaces to local area networks (LANs), wide area networks (WANs), wireless networks, the Internet, and blockchains.

In some embodiments, the healthcare management system 140 is operable on one or more processors and processing support modules such as data busses, memory device(s), mass storage device(s), and I/O device(s) to communicate with one other processing support modules, as well as other coupled devices. Busses can include one or more of several types of bus structures, such as a system bus, graphics bus, PCI bus, IEEE 1394 bus, or a USB bus. Using the processors and processing support modules, healthcare management system 140 can execute programs or applications to provide for data capture, receipt, analysis, and transmission.

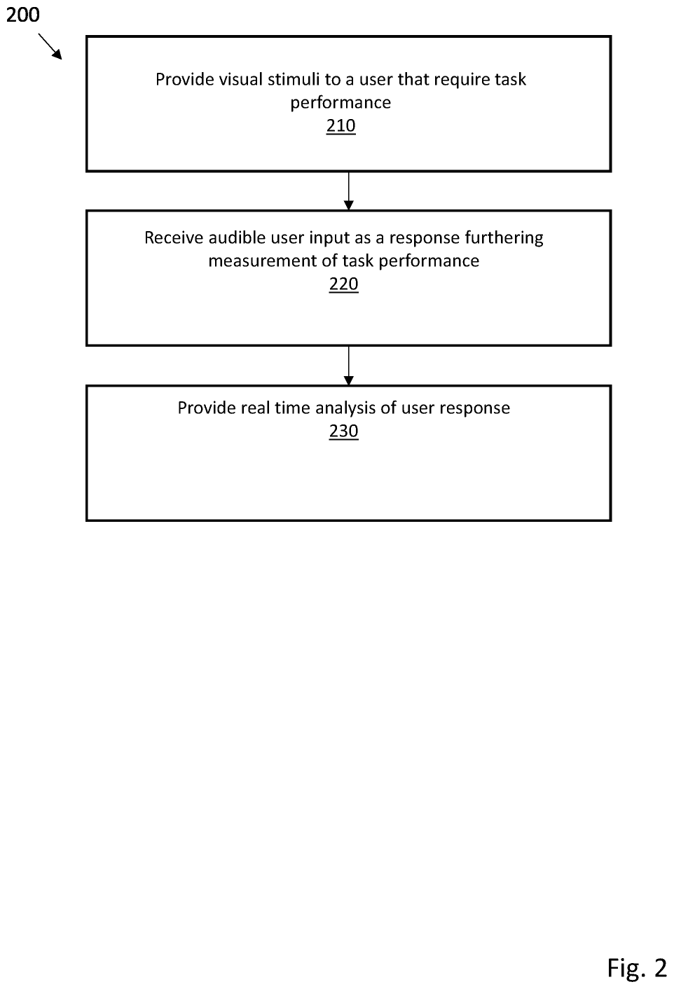

FIG. 2 illustrates a method 200 forming an element of a cognitive training methodology that includes a device to repeatedly present visual stimuli to a user that require performance of a task (step 210). A microphone can be positioned to provide audio input from the user to the device, with the audio input from the user providing input required to measure task performance (step 220). Real time analysis of measured task performance can be provided to the user (step 230).

For purposes of illustration, the described systems and methods include programs and other executable program components that are shown herein as discrete blocks, although it is understood that such programs and components may reside at various times in different storage components of local, server based, or cloud computing based systems and are executed by processor(s). Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter is described in language specific to structural features and/or methodological acts, it is to be understood that the subject matter defined in the appended claims is not necessarily limited to the described features or acts described herein. Rather, the described features and acts are disclosed as example forms of implementing the claims.

Those skilled in the art will appreciate that the disclosure may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote processing systems or memory storage devices. In some embodiments, more advanced procedures for providing tracking and data management services can be used, including use of blockchain based procedures.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

It should be noted that the sensor embodiments discussed herein may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the disclosure are directed to computer program products comprising such logic (e.g., in the form of software) stored on any computer useable medium. Such software, when executed in one or more data processing devices, causes a device to operate as described herein.

While various embodiments of the present disclosure are described herein, it should be understood that they are presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the described exemplary embodiments. The description herein is presented for the purposes of illustration and description. It is not intended to be exhaustive or to limit the disclosure to the precise form disclosed. Many modifications and variations are possible in light of the disclosed teaching. Further, it should be noted that any or all of the alternate implementations discussed herein may be used in any combination desired to form additional hybrid implementations of the disclosure.

1. A cognitive training system, comprising
a device to repeatedly present visual stimuli to a user that require performance of a task;
a microphone positioned to provide audio input from the user to the device, with the audio input from the user providing input required to measure task performance; and
a processing system to perform real-time analysis of measured task performance.

2. The cognitive training system of claim 1, wherein the device is any of smart watch, a wearable device, a smartphone, a desktop computer, a notebook computer, a tablet computer, a server computer, a handheld computer, one or more imagers, and a digital camera.

3. The cognitive training system of claim 1, wherein the task is a cognitive game that accepts voice commands.

4. The cognitive training system of claim 1, wherein the real-time analysis includes parsing the audio input into one or more words.

5. The cognitive training system of claim 1, wherein the processing system determines whether the audio input corresponds to one or more legal task responses.

6. The cognitive training system of claim 1, wherein a goal of the task includes the user responding with any combination of a number input, a location of one or more stimuli on a screen associated with the device, and an identity of a visual or auditory stimulus displayed on the screen.

7. The cognitive training system of claim 1, wherein a goal of the task includes the user inputting a word that fits all of the requirements of one or more stated rules of the task.

8. The cognitive training system of claim 1, wherein a goal of the task includes the user responding with a number that fits all of the requirements of one or more stated rules of the task.

9. The cognitive training system of claim 1, wherein a goal of the task includes the user spelling a word.

10. The cognitive training system of claim 1, further comprising a healthcare management system communicatively coupled to the device via a communication network, wherein the healthcare management system is configured to individually or collectively provide medical data collection and analysis services, along with support for remote clinical trials and clinical care.

11. A method comprising:
providing visual stimuli to a user that require performance of a task;
receiving audible user input as a response furthering measurement of task performance; and
providing real-time analysis of the response.

12. The method of claim 11, wherein the task is a cognitive game that accepts voice commands.

13. The method of claim 11, further comprising parsing the audio input into one or more words.

14. The method of claim 11, further comprising determining whether the audio input corresponds to one or more legal task responses.

15. The method of claim 11, wherein a goal of the task includes the user responding with any combination of a number input, a location of one or more stimuli on a screen associated with the device, and an identity of a visual or auditory stimulus displayed on the screen.

16. The method of claim 11, wherein a goal of the task includes the user inputting a word that fits all of the requirements of one or more stated rules of the task.

17. The method of claim 11, wherein a goal of the task includes the user responding with a number that fits all of the requirements of one or more stated rules of the task.

18. The method of claim 11, wherein a goal of the task includes the user spelling a word.

19. The method of claim 11, further comprising creating a phonological loop, wherein the phonological loop stores language in a short-term memory store.

20. The method of claim 11, further comprising extracting, from the audible user input, prosody and pace.
